SEQUENCE LISTING (3 sequences)

SEQ ID NO: 1 - protein, 233 aa, Porcine circovirus 2
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP

SEQ ID NO: 2 - protein, 234 aa, Porcine circovirus 2
MTYPRRRFRRRRHRPRSHLGQILRRRPWLVHPRHRYRWRRKNGIFNTRLSRTIGYTVKKTTVTTPSWNVDMMRFNINDFLPPGGGSNPLTVPFEYYRIRKVKVEFWPCSPITQGDRGVGSTAVILDDNFVTKANALTYDPYVNYSSRHTITQPFSYHSRYFTPKPVLDRTIDYFQPNNKRNQLWLRLQTTGNVDHVGLGTAFENSIYDQDYNIRITMYVQFREFNLKDPPLNPK

SEQ ID NO: 3 - protein, 579 aa, Porcine parvovirus
MSENVEQHNPINAGTELSATGNESIGGGGGGGGRGSIGVGVSTGSFNNQTEFQYLGEGLVRITAHASRLIHLNMPEHETYKRIHVLNSESGVAGQMVQDDAHTQMVTPWSLIDANAWGVWFNPADWQLISNNMTEINLVSFEQEIFNVVLKTITESATSPPTKIYNNDLTASLMVALDTNNTLPYTPAAPRSETLGFYPWLPTKPTQYRYYLSCTRNLNPPTYTGQSEQITDSIQTGLHSDIMFYTIENAVPIHLLRTGDEFSTGIYHFDTKPLKLTHSWQTNRSLGLPPKLLTEPTTEGDQHPGTLPAANTRKGYHQTINNSYTEATAIRPAQVGYNTPYMNFEYSNGGPFLTPIVPTADTQYNDDEPNGAIRFTMGYQHGQLTTSSQELERYTFNPQSKCGRAPKQQFNQQSPLNLQNTNNGTLLPSDPIGGKTNMHFMNTLNTYGPLTALNNTAPVFPNGQIWDKELDTDLKPRLHVTAPFVCKNNPPGQLFVKIAPNLTDDFNADSPQQPRIITYSNFWWKGTLTFTAKMRSSNMWNPIQQHTTTAENIGNYIPTNIGGIKMFPEYSQLIPRKLY

(19) United States
(12) Patent Application Publication
(10) Pub. No.: US 2026/0306617 A1
(43) Pub. Date: Oct. 8, 2026

(54) METHOD FOR INDUCING AN IMMUNE RESPONSE

(71) Applicant: ACCLARENT, INC., Irvine, CA (US)

(72) Inventors: Fernando Lopes Leivas LEITE, Atlanta, GA (US); Marc Eichmeyer, Bondurant, IA (US); Abby PATTERSON, Story City, IA (US)

(21) Appl. No.: 19/480,051

(22) PCT Filed: May 2, 2024
(86) PCT No.: PCT/IB2024/052471

§ 371 Date: Oct. 30, 2025

Related U.S. Application Data
(60) Provisional application No. 63/499,941, filed on May 3, 2023.

(51) Int. Cl. A61K 39/23; A61K 9/00; A61K 39/00

(52) U.S. Cl. CPC A61K 39/23; A61K 9/0095; A61K 2039/5258; A61K 2039/542; A61K 2039/545; A61K 2039/552

(57) ABSTRACT

The present invention relates to methods for inducing an immune response in a subject, the method comprising administering orally to the subject an immunogenic composition comprising non-replicating virus antigen. In one example, an immunogenic composition is administered via the oral route to a pig, wherein the immunogenic composition comprises a recombinant virus capsid protein capable of forming a virus-like particle. This allows to simply induce an immune response in the pigs, while simultaneously reducing the propensity for adverse reactions, eliminating the risk of injection site reactions, avoiding pain, and putting less stress on the animals.

### PRIORITY

This application claims priority to U.S. Provisional Pat. App. No. 63/467,679, entitled “Guide Rail Actuation Assembly for Balloon Dilation Instrument,” filed May 19, 2023, the disclosure of which is incorporated by reference herein, in its entirety.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pat. No. 11,534,192, entitled “Methods and Apparatus for Treating Disorders of the Sinuses,” issued Dec. 27, 2022, the disclosure of which is incorporated by reference herein, in its entirety; U.S. Pat. No. 9,579,448, entitled “Balloon Dilation Catheter System for Treatment and Irrigation of the Sinuses,” issued Feb. 28, 2017, the disclosure of which is incorporated by reference herein, in its entirety; U.S. Pat. No. 9,155,492, entitled “Sinus Illumination Lightwire Device,” issued Oct. 13, 2015, the disclosure of which is incorporated by reference herein, in its entirety; and U.S. Pub. No. 2021/0361912, entitled “Shaft Deflection Control Assembly for ENT Guide Instrument,” published Nov. 25, 2021, the disclosure of which is incorporated by reference herein, in its entirety.

In the context of Eustachian tube dilation, a dilation catheter or other dilation instrument may be inserted into the Eustachian tube and then be inflated or otherwise expanded to thereby dilate the Eustachian tube. The dilated Eustachian tube may provide improved ventilation from the nasopharynx to the middle ear and further provide improved drainage from the middle ear to the nasopharynx. Methods and devices for dilating the Eustachian tube are disclosed in U.S. Pat. No. 10,206,821, entitled “Eustachian Tube Dilation Balloon with Ventilation Path,” issued Feb. 19, 2019, the disclosure of which is incorporated by reference herein, in its entirety; and U.S. Pat. No. 11,013,896, entitled “Method and System for Eustachian Tube Dilation,” issued May 25, 2021, the disclosure of which is incorporated by reference herein, in its entirety.

In some scenarios, it may be desirable to provide adjustability to a medical instrument, to allow the same medical instrument to readily access different anatomical structures. For instance, it may be desirable to provide a dilation instrument with an adjustable guide that facilitates access to Eustachian tubes and different passageways associated with drainage of paranasal sinuses. Examples of dilation instruments with adjustable guides are described in U.S. Pat. No. 10,137,285, entitled “Balloon Dilation System with Malleable Internal Guide,” issued Nov. 27, 2018, the disclosure of which is incorporated by reference herein, in its entirety; U.S. Pat. No. 11,013,897, entitled “Apparatus for Bending Malleable Guide of Surgical Instrument,” issued May 25, 2021, the disclosure of which is incorporated by reference herein, in its entirety; and U.S. Pat. No. 11,534,192, entitled “Methods and Apparatus for Treating Disorders of the Sinuses,” issued Dec. 27, 2022, the disclosure of which is incorporated by reference herein, in its entirety. While several systems and methods have been made and used to dilate anatomical passageways within a patient, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1A depicts a perspective view of an example of a medical instrument, with a dilation catheter in a proximal position and a guide rail in a proximal position;

FIG. 1B depicts a perspective view of the medical instrument of FIG. 1A, with the dilation catheter in a distal position and the guide rail in the proximal position;

FIG. 2 depicts a perspective view of the medical instrument of FIG. 1A, with the dilation catheter in the proximal position and the guide rail in a distal position.

FIG. 3A depicts a perspective view of a distal portion of the shaft assembly of the medical instrument of FIG. 1A, with the guide rail in a bent state and at a first angular position;

FIG. 3B depicts a perspective view of the distal portion of FIG. 3A, with the guide rail in the bent state and at a second angular position;

FIG. 4 depicts a perspective view of a guide rail actuator of the medical instrument of FIG. 1A;

FIG. 5 depicts a perspective view of a proximal portion of a housing of a body assembly of the medical instrument of FIG. 1A;

FIG. 6A depicts a cross-sectional side view of a proximal portion of the body assembly of the medical instrument of FIG. 1A, with the guide rail actuator and the guide rail in a proximal position;

FIG. 6B depicts a cross-sectional side view of the proximal portion of FIG. 6A, with the guide rail actuator and the guide rail in a distal position;

FIG. 7 depicts a perspective view of another example of a medical instrument, with a dilation catheter in a proximal position and a guide rail in a distal position;

FIG. 8 depicts a perspective view of a dilation catheter actuation assembly of the medical instrument of FIG. 7 and a guide rail actuation assembly of the medical instrument of FIG. 7;

FIG. 9 depicts a cross-sectional perspective view of a manifold body of the dilation catheter actuation assembly of FIG. 8;

FIG. 10 depicts a perspective view of a block of the guide rail actuation assembly of FIG. 8;

FIG. 11A depicts a cross-sectional side view of the medical instrument of FIG. 7, with the guide rail in a proximal position and the dilation catheter in the proximal position;

FIG. 11B depicts a cross-sectional side view of the medical instrument of FIG. 7, with the guide rail in the distal position and the dilation catheter in the proximal position;

FIG. 11C depicts a cross-sectional side view of the medical instrument of FIG. 7, with the guide rail in the distal position and the dilation catheter in a distal position;

FIG. 12A depicts a perspective view of an example of an alternative body assembly that may be integrated into a medical instrument, with a nose body in a proximal position;

FIG. 12B depicts a perspective view of the body assembly of FIG. 12A, with the nose body in a distal position;

FIG. 13 depicts an end elevation view of the nose body of FIG. 12A;

FIG. 14 depicts a cross-sectional side view of the nose body of FIG. 12A;

FIG. 15 depicts a perspective view of a handle of the body assembly of FIG. 12A;

FIG. 16 depicts a perspective view of a driver of the body assembly of FIG. 12A;

FIG. 17A depicts a cross-sectional side view of the body assembly of FIG. 12A, with the nose body and driver body in a proximal position, and with a dilation catheter actuation rack in a proximal position;

FIG. 17B depicts a cross-sectional side view of the body assembly of FIG. 12A, with the nose body and driver body in a distal position, and with a dilation catheter actuation rack in the proximal position;

FIG. 17C depicts a cross-sectional side view of the body assembly of FIG. 12A, with the nose body and driver body in a distal position, and with a dilation catheter actuation rack in a distal position;

FIG. 18A depicts a perspective view of another example of a medical instrument, with a guide rail in a distal position; and

FIG. 18B depicts a perspective view of the medical instrument of FIG. 18A, with the guide rail in a proximal position.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Examples of Dilation Instruments with Adjustable Guide Rails

In some scenarios, it may be desirable to advance a dilation catheter into an anatomical passageway in or near the ear, nose, or throat of a patient; and expand the dilator to thereby dilate the passageway. For instance, it may be desirable to dilate a paranasal sinus ostium or other passageway associated with drainage of a paranasal sinus cavity, a Eustachian tube, a stenotic region in an airway of a patient, etc. It may also be desirable to provide a single instrument that is capable of dilating different anatomical passageways in a patient. To facilitate such capabilities, the dilation instrument may include an adjustable guide, such as a malleable guide. To the extent that a malleable guide may facilitate different bend angles in a guide, it may be further desirable to achieve different longitudinal positions and/or angular positions in a guide, to thereby further facilitate access to different anatomical passageways. The following describes examples of dilation instruments with guides having a combination of adjustable bend angles, adjustable longitudinal positions, and/or adjustable angular positions.

A. Dilation Instrument with Malleable Guide Rail having Adjustable Longitudinal and Angular Positions; and Translating Dilation Catheter Actuator

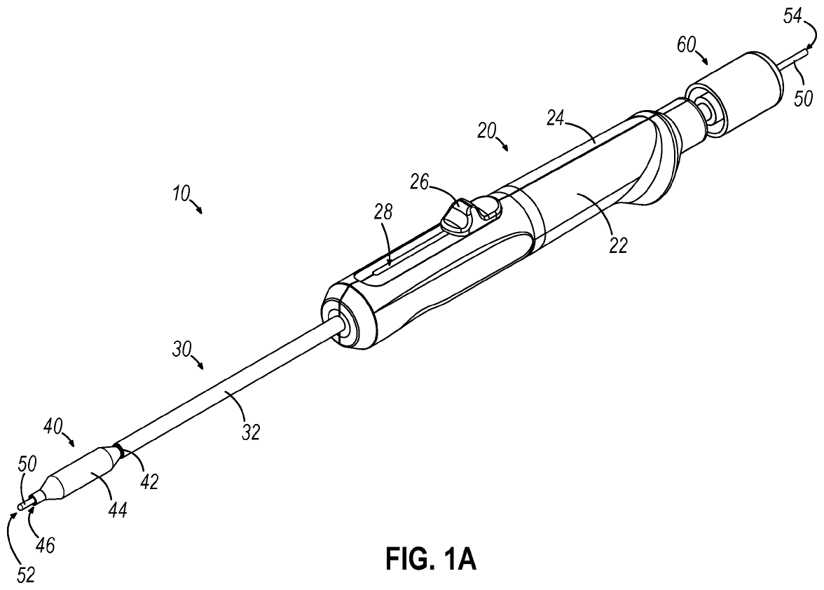

FIGS. 1A-6B show an example of a dilation instrument (10) that may be used to dilate an anatomical passageway in or near an ear, nose, or throat of a patient. Instrument (10) of this example includes a body assembly (20) and a shaft assembly (30). Body assembly (20) includes a first housing (22), a second housing (24), and a dilation catheter actuator (26). Housings (22, 24) cooperate to form a handle. Dilation catheter actuator (26) is slidably disposed in a slot (28) formed in the handle. Dilation catheter actuator (26) is operable to drive longitudinal movement of a dilation catheter (40) as described in greater detail below.

Shaft assembly (30) includes an outer sheath (32), a guide rail (50), and dilation catheter (40). In the present example, these components are configured to be positioned coaxially with each other, such that outer sheath (32) is positioned externally, dilation catheter (40) is positioned internal to outer sheath (32), and guide rail (50) is positioned internal to dilation catheter (40). Outer sheath (32) of the present example is rigid, though other versions may be malleable or otherwise flexible. In the present example, outer sheath (32) does not enter the head of the patient during operation of instrument (10), though some scenarios may exist where outer sheath (32) enters the patient during operation of instrument (10).

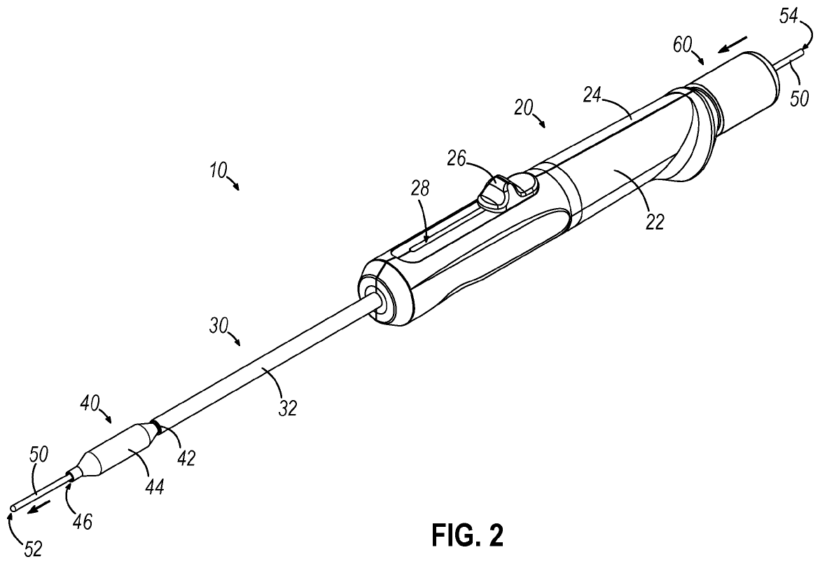

Guide rail (50) of the present example is malleable and has an atraumatic distal tip (52). In some versions, distal tip (52) is dome shaped. In some other versions, distal tip (52) is enlarged (e.g., configured as a ball tip or blueberry tip, etc.). As shown in FIGS. 3A-3B, the malleability of guide rail (50) allows guide rail (50) to be bent to a desired bend angle before being inserted into the head of the patient. The malleability of guide rail (50) may allow guide rail (50) to maintain the bend angle of a bend (56) while guide rail (50) is disposed in the head of the patient, including while dilation catheter (40) is advanced distally relative to guide rail (50). Such operability of guide rail (50) may promote access by dilation catheter (40) to various locations within the head of a patient, such as the maxillary sinus ostium, the frontal recess, the sphenoid sinus ostium, the Eustachian tube, etc., based on the selected bend angle. By way of example only, the bending of guide rail (50) may be performed in accordance with at least some of the teachings of U.S. Pat. No. 11,013,897, entitled “Apparatus for Bending Malleable Guide of Surgical Instrument,” issued May 25, 2021, the disclosure of which is incorporated by reference herein, in its entirety.

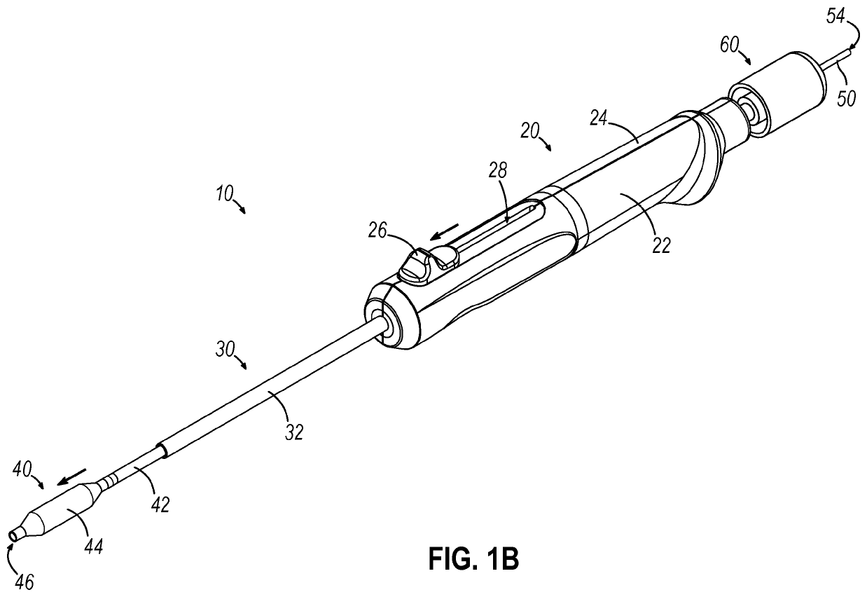

Dilation catheter (40) of the present example includes a shaft (42) having an integral balloon (44) and a distal tip (46). Shaft (42) is coaxially positioned with outer sheath (32) and guide rail (50). Shaft (42) defines two inner lumens, including a first lumen in which guide rail (50) is slidably disposed and a second lumen in fluid communication with balloon (44). Balloon (44) may comprise a non-extensible material and may be sized and configured to fit within a targeted anatomical passageway while in the deflated state; then dilate the targeted anatomical passageway while in the inflated state. It should be understood that balloon (44) is shown as being inflated in FIGS. 1A-2 for schematic purposes only; and that balloon (44) may in fact be deflated at such stages of operation.

Shaft (42) is fixedly coupled with dilation catheter actuator (26) such that dilation catheter actuator (26) is operable to drive dilation catheter (40) longitudinally between a proximal position (FIG. 1A) and a distal position (FIG. 1B). In some versions, shaft (42) is coupled with dilation catheter actuator (26) directly; while in other versions shaft (42) is coupled with dilation catheter actuator (26) indirectly (e.g., via one or more intervening components). In the present example, distal tip (46) is proximal to distal tip (52) when dilation catheter (40) is in the proximal position (FIG. 1A); and distal to distal tip (52) when dilation catheter (40) is in the distal position (FIG. 1B). In some other versions, shaft assembly (30) is configured such that distal tip (46) remains proximal to distal tip (52) even when dilation catheter (40) is in the distal position.

One or both of distal tips (46, 52) may comprise an indicator element. Each such indicator element may comprise a position sensor and/or an illuminating feature. In versions where an indicator element includes a position sensor, the position sensor may comprise one or more coils that generate signals in response to electromagnetic fields emitted by magnetic field generators of an image guided surgery system that is operable to determine the real-time position of the position sensor in three-dimensional space based on the signals generated by the position sensor. In versions where an indicator element includes an illuminating feature, the illuminating feature may be operable to project light outwardly from indicator element. Such an illuminating feature may provide transillumination through the skin of the patient. In some such versions, the illuminating feature includes an optically transmissive window that is optically coupled with one or more optical fibers, with such one or more optical fibers being optically coupled with a light source. In some other versions, illuminating feature includes one or more LEDs or other local sources of light positioned locally at distal tip (46, 52).

As noted above, it may be desirable to rotate and/or translate guide rail (50) to provide further adjustability to the position and orientation of guide rail (50), which may in turn further facilitate access to different anatomical passageways. To that end, instrument (10) of the present example further includes a guide rail actuator (60) at the proximal end of body assembly (20). Guide rail actuator (60) is fixedly coupled with guide rail (50) such that guide rail actuator (60) is operable to drive guide rail (50) to a distal position as shown in FIG. 2; and to rotate guide rail (50) to different angular orientations as shown in FIGS. 3A-3B. In some versions, guide rail (50) is coupled with guide rail actuator (60) directly; while in other versions guide rail (50) is coupled with guide rail actuator (60) indirectly (e.g., via one or more intervening components). In the present example, guide rail (50) passes fully through guide rail actuator (60) such that a proximal end (54) of guide rail (50) is proximal to guide rail actuator (60); and such that the proximal portion of guide rail (50) is not disposed in the handle formed by housings (22, 24).

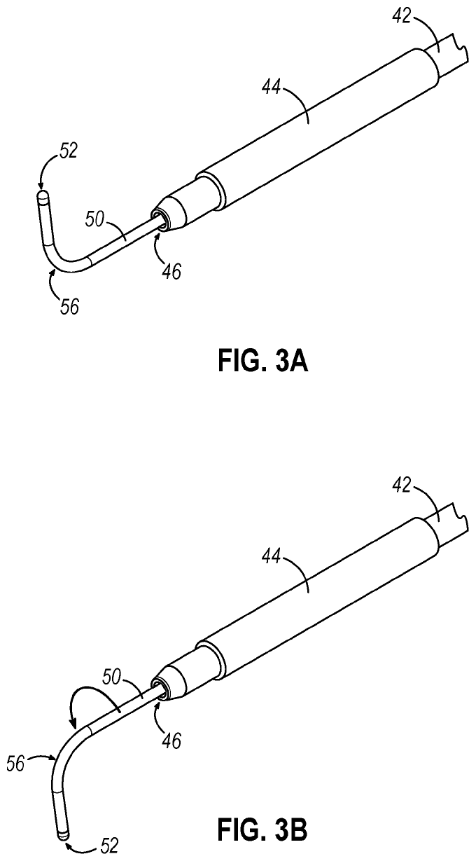

FIG. 4 shows guide rail actuator (60) in further detail. As shown, guide rail actuator (60) comprises a proximal knob (62) defining a recess (64), an outer shaft (66) extending distally from proximal knob (62), and an inner shaft (68) extending distally from outer shaft (66). Guide rail (50) is fixedly secured within inner shaft (68). A locking member (70) is fixedly secured to the distal end of outer shaft (66). Locking member (70) comprises an annular flange (72) having a radial notch (74) and a radial protrusion (76) adjacent to radial notch (74). While locking member (70) of the present example only has one radial notch (74), other versions may include two or more radial notches (74) at different angular positions about annular flange (72). Similarly, while locking member (70) of the present example only has one radial protrusion (76), other versions may include two or more radial protrusions (76) at different angular positions about annular flange (72).

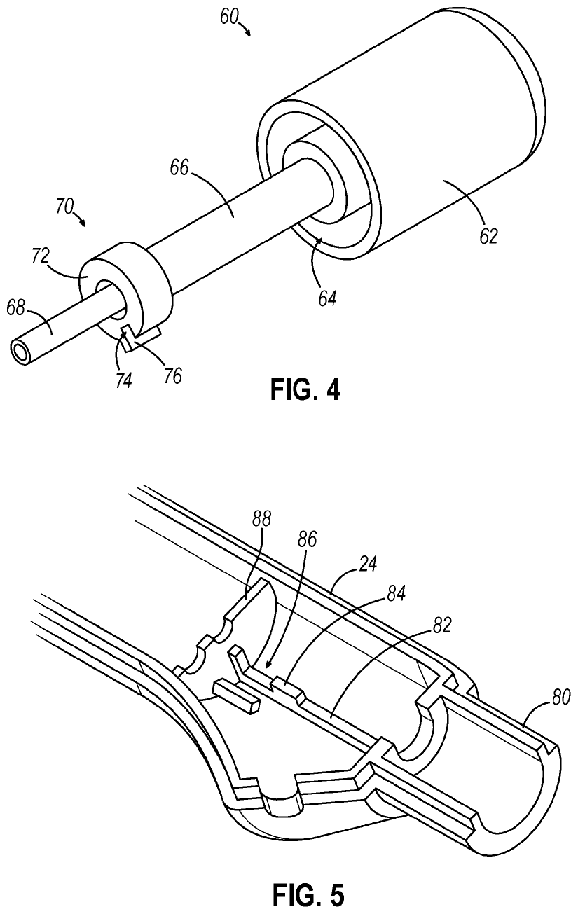

FIG. 5 shows an interior region of a proximal portion of housing (24). As shown, housing (24) includes a proximally extending cylindrical feature (80) that is configured to be freely positioned within recess (64) of proximal knob (62). Housing (24) also includes a longitudinally extending inner wall (82) with an inwardly oriented projection (84) and a distal region (86) that is distal to projection (84). A transversely extending inner wall (88) is just distal to distal region (86) of inner wall (82).

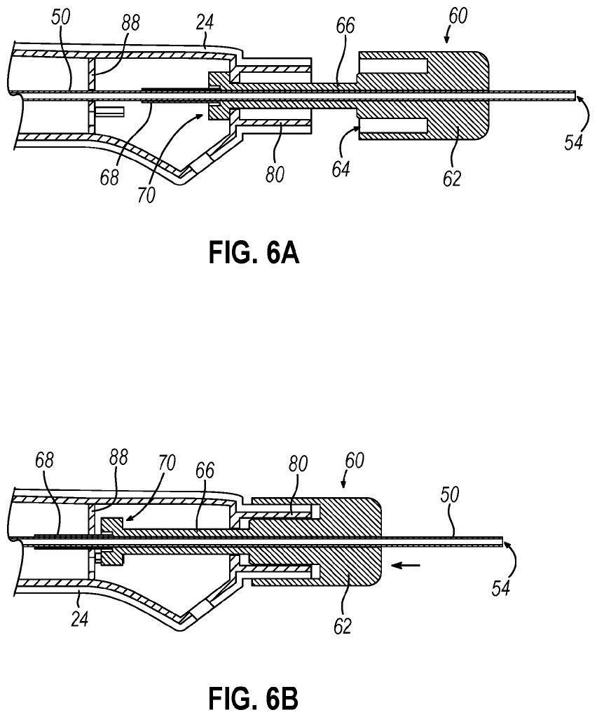

In the present example, projection (84) is sized to fit in radial notch (74) and thereby prevent rotation of locking member (70) relative to housing (24). In addition, or in the alternative, projection (84) may be configured to engage radial protrusion (76) to prevent rotation of locking member (70) relative to housing (24). To selectively disengage notch (74) and/or radial protrusion (76) from projection (84), the operator may push guide rail actuator (60) distally as shown in FIG. 6B. While guide rail actuator (60) is in the distal position, the operator may rotate guide rail actuator (60) to thereby change the angular orientation of guide rail (50), then return guide rail actuator (60) to the proximal position as shown in FIG. 6A. In some versions, a resilient member (e.g., spring, etc.) is interposed between inner wall (88) and annular flange (72) to resiliently bias guide rail actuator (60) proximally to the proximal position shown in FIG. 6A. Also in some versions, when notch (74) and/or radial protrusion (76) are engaged with projection (84) while guide rail actuator (60) is in the proximal position, this engagement fixes the angular position of guide rail (50); whereas guide rail (50) is free to rotate through some range of angular motion when notch (74) and/or radial protrusion (76) are disengaged from projection (84) while guide rail actuator (60) is in the proximal position. The operator may thus choose to engage notch (74) and/or radial protrusion (76) with projection (84) when guide rail actuator (60) is in the distal position; or disengage these features when guide rail actuator (60) is in the distal position.

In some other versions, locking member (70) may include a plurality of notches (74) and/or radial protrusions (76), such that the operator may engage a selected one of the notches (74) and/or radial protrusions (76) with projection (84) when guide rail actuator (60) is in the distal position, to thereby select a desired angular orientation for guide rail (50). When guide rail actuator (60) is returned to the proximal position, guide rail (50) may be effectively locked at the selected angular orientation due to the engagement between the selected notch (74) and/or radial protrusion (76) with projection (84). As another variation, the interior of the handle formed by housings (22, 24) may include two or more projections (84) (in inner regions of one or both of housings (22, 24)), such that the operator may engage notch (74) and/or radial protrusion (76) with a selected one of the projections (84) when guide rail actuator (60) is in the distal position, to thereby select a desired angular orientation for guide rail (50). When guide rail actuator (60) is returned to the proximal position, guide rail (50) may be effectively locked at the selected angular orientation due to the engagement between notch (74) and/or radial protrusion (76) with the selected projection (84).

While FIGS. 1A-1B show dilation catheter (40) being advanced distally along a straight guide rail (50), dilation catheter (40) may also be advanced distally along a guide rail (50) having a bend (56) formed therein; with guide rail (50) being rotated to any selected angular orientation (e.g., as shown in FIGS. 3A-3B). At the distal position, balloon (44) may be inflated to dilate the targeted anatomical passageway.

B. Dilation Instrument with Malleable Guide Rail having Proximally-Adjustable Longitudinal Position; and Rotary Dilation Catheter Actuator

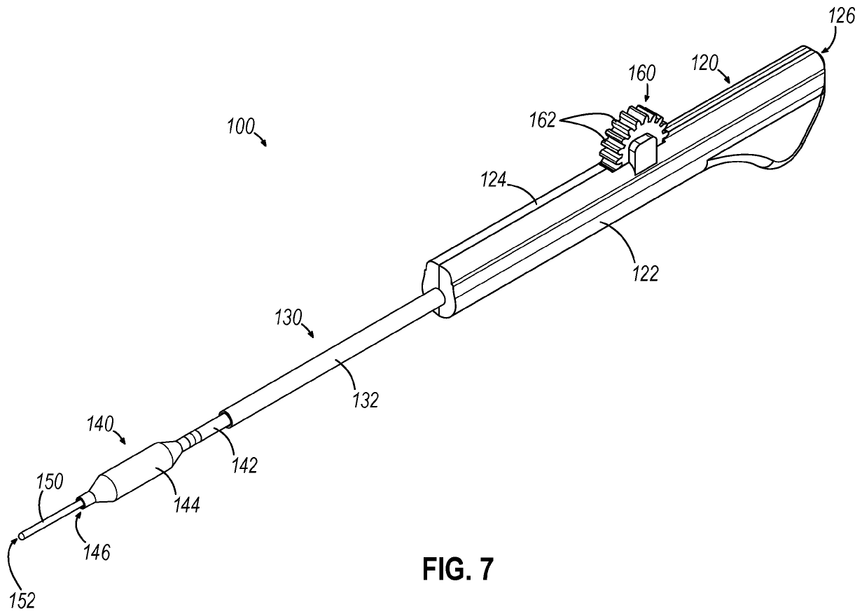

FIGS. 7-11C show another example of a dilation instrument (100) that may be used to dilate an anatomical passageway in or near an ear, nose, or throat of a patient. Instrument (100) of this example includes a body assembly (120) and a shaft assembly (130). Body assembly (120) includes a first housing (122), a second housing (124), and a dilation catheter actuator (160). Housings (122, 124) cooperate to form a handle. Dilation catheter actuator (160) is rotatably supported by the handle. Dilation catheter actuator (160) is operable to drive longitudinal movement of a dilation catheter (140) as described in greater detail below.

Shaft assembly (130) includes an outer sheath (132), a guide rail (150), and dilation catheter (140). In the present example, these components are configured to be positioned coaxially with each other, such that outer sheath (132) is positioned externally, dilation catheter (140) is positioned internal to outer sheath (132), and guide rail (150) is positioned internal to dilation catheter (140). Outer sheath (132) of the present example is rigid, though other versions may be malleable or otherwise flexible. In the present example, outer sheath (132) does not enter the head of the patient during operation of instrument (100), though some scenarios may exist where outer sheath (132) enters the patient during operation of instrument (100).

Guide rail (150) of the present example is malleable and has an atraumatic distal tip (152). In some versions, distal tip (152) is dome shaped. In some other versions, distal tip (152) is enlarged (e.g., configured as a ball tip or blueberry tip, etc.). The malleability of guide rail (150) may allow guide rail (150) to maintain the bend angle of a bend while guide rail (150) is disposed in the head of the patient, including while dilation catheter (140) is advanced distally relative to guide rail (150). Such operability of guide rail (150) may promote access by dilation catheter (140) to various locations within the head of a patient, such as the maxillary sinus ostium, the frontal recess, the sphenoid sinus ostium, the Eustachian tube, etc., based on the selected bend angle. By way of example only, the bending of guide rail (150) may be performed in accordance with at least some of the teachings of U.S. Pat. No. 11,013,897, entitled “Apparatus for Bending Malleable Guide of Surgical Instrument,” issued May 25, 2021, the disclosure of which is incorporated by reference herein, in its entirety.

Dilation catheter (140) of the present example includes a shaft (142) having an integral balloon (144) and a distal tip (146). Shaft (142) is coaxially positioned with outer sheath (132) and guide rail (150). Shaft (142) defines two inner lumens, including a first lumen in which guide rail (150) is slidably disposed and a second lumen in fluid communication with balloon (144). Balloon (144) may comprise a non-extensible material and may be sized and configured to fit within a targeted anatomical passageway while in the deflated state; then dilate the targeted anatomical passageway while in the inflated state. It should be understood that balloon (144) is shown as being inflated in FIGS. 7-8 and 11A-11C for schematic purposes only; and that balloon (144) may in fact be deflated at such stages of operation.

One or both of distal tips (146, 152) may comprise an indicator element. Each such indicator element may comprise a position sensor and/or an illuminating feature. In versions where an indicator element includes a position sensor, the position sensor may comprise one or more coils that generate signals in response to electromagnetic fields emitted by magnetic field generators of an image guided surgery system that is operable to determine the real-time position of the position sensor in three-dimensional space based on the signals generated by the position sensor. In versions where an indicator element includes an illuminating feature, the illuminating feature may be operable to project light outwardly from indicator element. Such an illuminating feature may provide transillumination through the skin of the patient. In some such versions, the illuminating feature includes an optically transmissive window that is optically coupled with one or more optical fibers, with such one or more optical fibers being optically coupled with a light source. In some other versions, illuminating feature includes one or more LEDs or other local sources of light positioned locally at distal tip (146, 152).

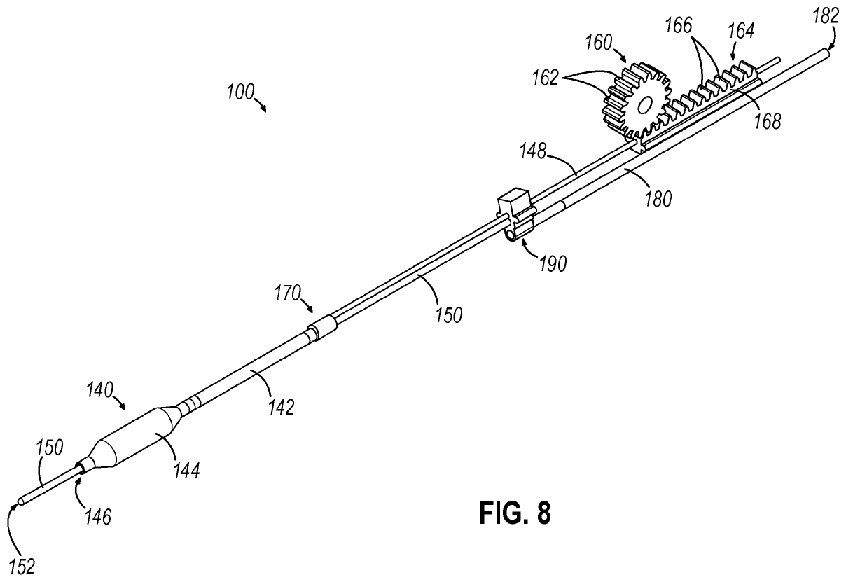

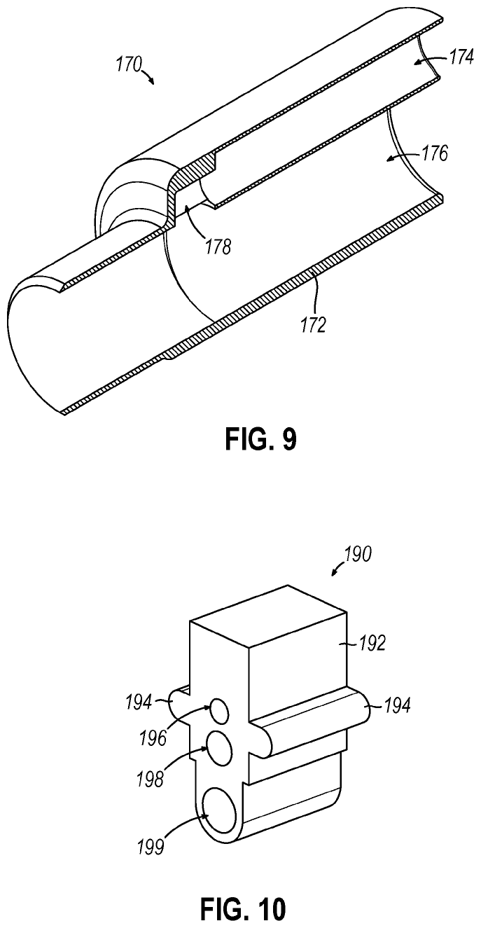

FIGS. 8-11C show features that are operable to drive translation of dilation catheter (140). In particular, FIG. 8 shows dilation catheter actuator (160) being in the form of a pinion with teeth (162) that mesh with corresponding teeth (166) of a rack (164). A drive rod (158) is fixedly secured to rack (164). Drive rod (158) is parallel to, and laterally offset from, shaft (142) and guide rail (150). A manifold (170) is fixedly secured to the distal end of drive rod (158). Shaft (142) is also fixedly secured to manifold (170), such that rack (164), drive rod (158), manifold (170), and dilation catheter (140) translate together unitarily. As shown in FIG. 9, manifold (170) includes a body (172) defining an upper bore (174), a lower bore (172), and a transverse opening (178). In the present example, the distal end of drive rod (158) is fixedly secured in upper bore (174). Drive rod (158) further defines a lumen (not shown) that is in fluid communication with upper bore (174) and transverse opening (178). Shaft (142) includes an inflation lumen (not shown) that is in fluid communication with transverse opening (178) and with balloon (144). The proximal end of drive rod (158) may be coupled with a source of inflation fluid (e.g., saline, etc.), such that balloon (144) may be inflated by fluid communicated along the path formed by the lumen of drive rod, upper bore (174), transverse opening (178), and the inflation lumen of shaft (142). In addition to having the proximal end of shaft (142) secured therein, lower bore (172) is configured to slidably accommodate guide rail (150).

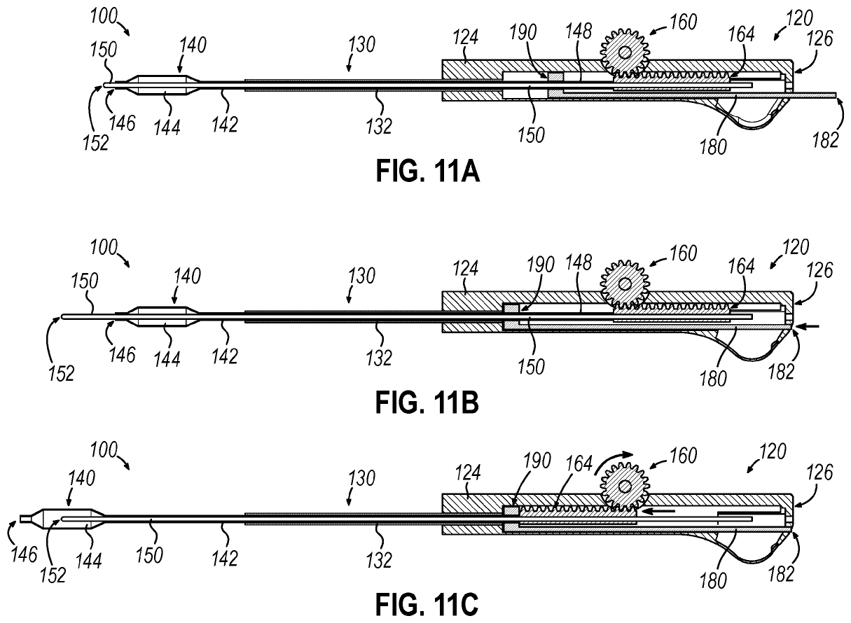

As noted above, it may be desirable to translate guide rail (150) to provide further adjustability to the position of guide rail (150). To that end, instrument (100) of the present example further includes a guide rail actuator (180) that is translatable relative to the handle formed by housings (122, 124). As shown in FIG. 8, guide rail actuator (180) of the present example is in the form of an elongate rod. A block (190) is fixedly secured to the distal end of guide rail actuator (180). The proximal end of guide rail (150) is also fixedly secured to block (190) such that guide rail (150), block (190), and guide rail actuator (180) translate unitarily with each other. As shown in FIG. 10, block (190) includes a body (192) having outwardly extending rails (194) and openings (196, 198, 199) formed therethrough. Rails (194) are disposed in corresponding channels (not shown) in housings (122, 124), such that housings (122, 124) support block (190) while allowing block (190) to translate relative to housings (122, 124). Drive rod (148) is slidably disposed in opening (196). The distal end of guide rail actuator (180) is fixedly disposed in opening (199). The proximal end of guide rail (150) is fixedly disposed in opening (198).

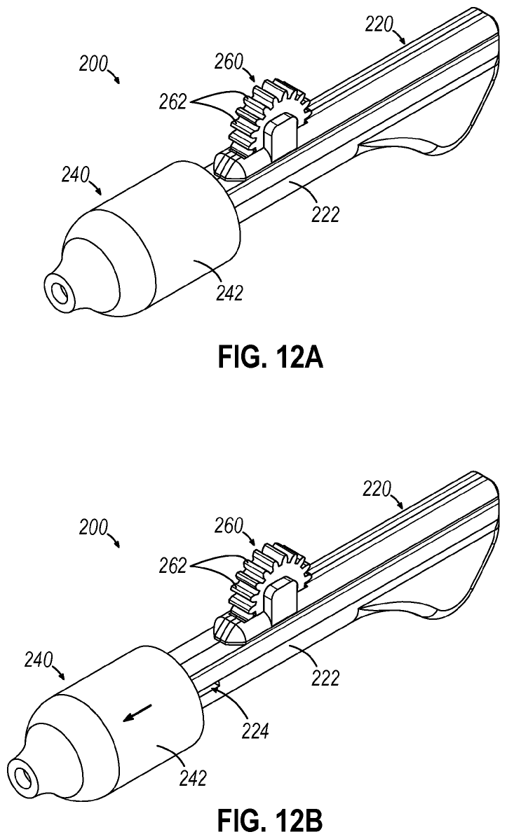

As best seen in FIG. 11A, an operational state of instrument (100) may begin with dilation catheter (140) and guide rail (150) both in proximal positions. In this state, a proximal end (182) of guide rail actuator (180) protrudes proximally from the proximal end (126) of the handle formed by housings (122, 124). Distal tip (152) of guide rail (150) is positioned distally in relation to distal tip (146) of dilation catheter (140). In some other versions, distal tip (152) of guide rail (150) is positioned proximally in relation to distal tip (146) of dilation catheter (140) in the initial state of operation. In the present example, block (190) is positioned to prevent rack (164) from advancing to a fully distal position, such that block (190) needs to be translated distally to accommodate full distal movement of rack (164). To that end, the operator may urge guide rail actuator (180) distally to the position shown in FIG. 11B. This distal movement of guide rail actuator (180) drives block (190) and guide rail (150) distally as shown. The distal positioning of block (190) provides clearance for rack (164) to advance to a fully distal position.

After guide rail (150) has been advanced distally as shown in the transition from FIG. 11A to FIG. 11B, the operator may advance dilation catheter (140) distally by rotating dilation catheter actuator (160). This may be accomplished using a finger or thumb of the same hand that grasps the handle formed by housings (122, 124). The rotation of dilation catheter actuator (160) drives rack (164) distally, due to meshing engagement between teeth (162, 166). The distal movement of rack (164) drives drive rod (148) and manifold (170) distally, thereby driving dilation catheter (140) distally as shown in FIG. 11C. While FIGS. 11A-11C show dilation catheter (140) being advanced distally along a straight guide rail (150), dilation catheter (140) may also be advanced distally along a guide rail (150) having a bend formed therein. At the distal position, balloon (144) may be inflated to dilate the targeted anatomical passageway.

In some versions, body assembly (120) further includes one or more resilient features, detents, and/or other features that prevent inadvertent distal movement of guide rail actuator (180) from the proximal position (FIG. 11A) to the distal position (FIGS. 11B-11C). Similarly, body assembly (120) may include one or more detents, latching features, or other latching features that maintain guide rail actuator (180) in the distal position after guide rail actuator (180) has been advanced to the distal position. In some such versions, such features may permanently maintain guide rail actuator (180) in the distal position after guide rail actuator (180). As another example, body assembly (120) may include selective locking/unlocking features that allow the operator to selectively transition guide rail actuator (180) between the distal position and the proximal position by pressing and releasing proximal end (182) in a manner similar to the pushbutton of a ballpoint pen (e.g., with rotary cam/thrust features, etc.).

In some cases it may be desirable to maintain guide rail (150) in the proximal position as shown in FIG. 11A up until an operator is ready to use instrument (100). By maintaining guide rail (150) in the proximal position before use, the positioning of all or nearly all of the distal portion of guide rail (150) within dilation catheter (140) may substantially reduce the risk of the distal portion of guide rail (150) getting inadvertently bent, damaged, etc.

C. Dilation Instrument with Malleable Guide Rail having Distally-Adjustable Longitudinal Position; and Rotary Dilation Catheter Actuator

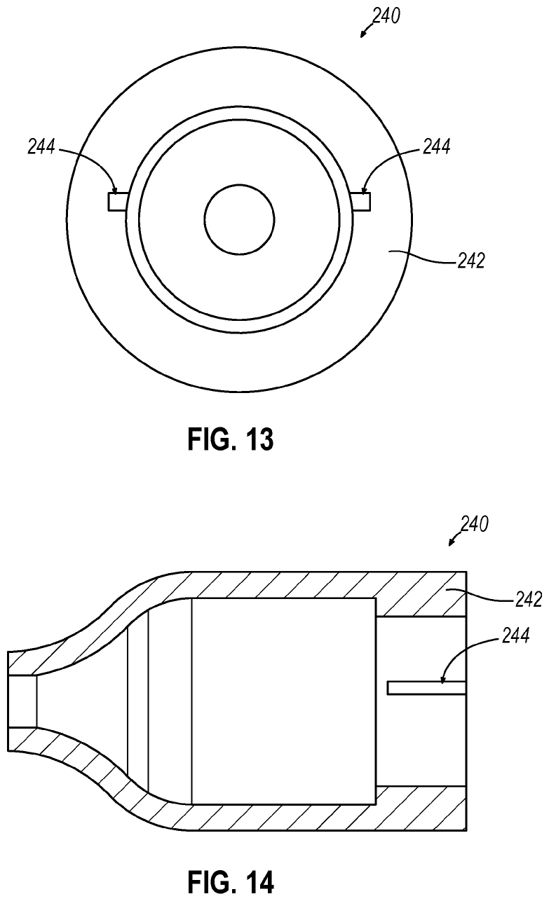

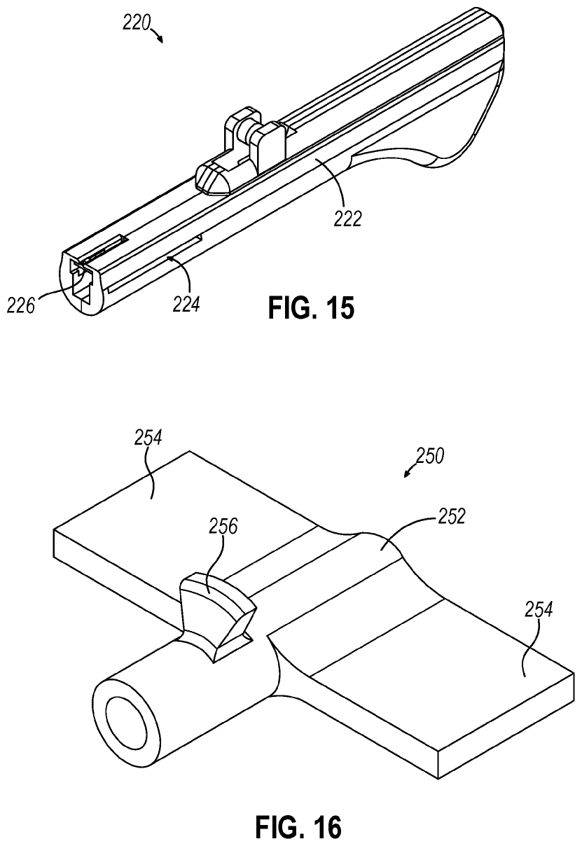

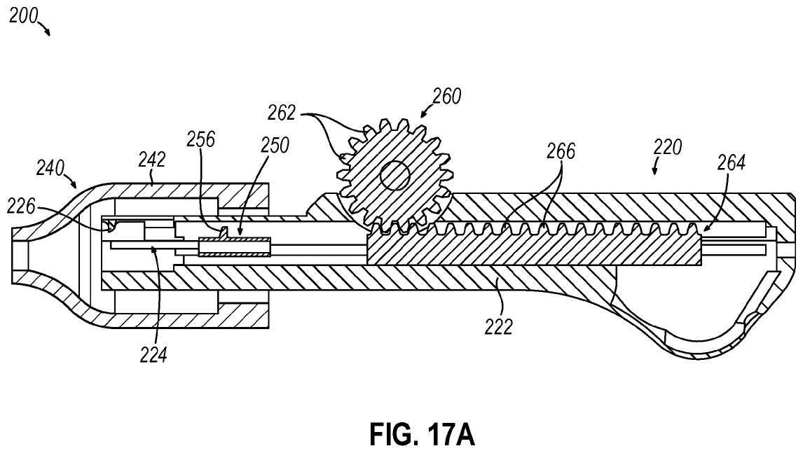

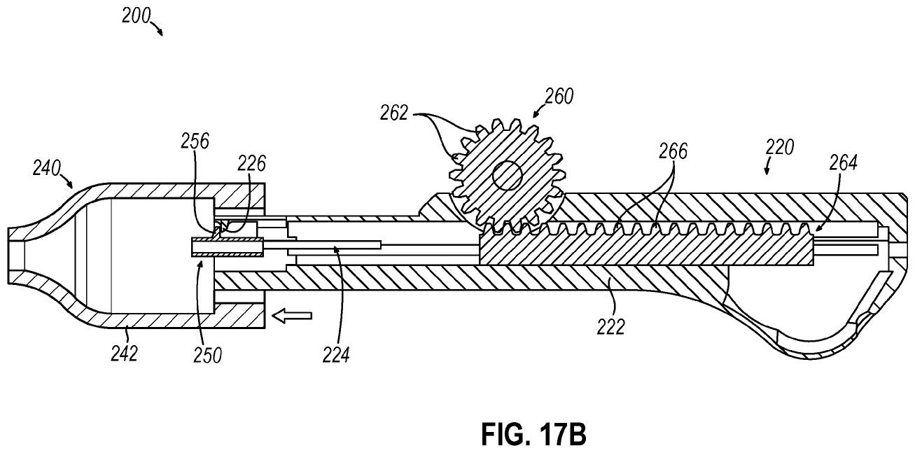

FIGS. 12A-17C show an example of an alternative body assembly (200) that may be incorporated into a dilation instrument like dilation instruments (10, 100) described above. Body assembly (200) of this example comprises a handle (220) formed by a housing (222). A dilation catheter actuator (260) is rotatably supported by housing (222). A guide rail actuator (240) is positioned distally relative to handle (220). As shown in FIGS. 12A-12B, guide rail actuator (240) is translatable between a proximal position (FIG. 12A) and a distal position (FIG. 12B). As shown in FIGS. 13-14, guide rail actuator (240) comprises a body (242) having a pair of internal notches (244). As shown in FIG. 15, handle (220) further includes a distal opening (226) and a pair of lateral slots (224), with lateral slots (224) being formed on each side of handle (220). A latch projection (226) extends downwardly in opening (226).

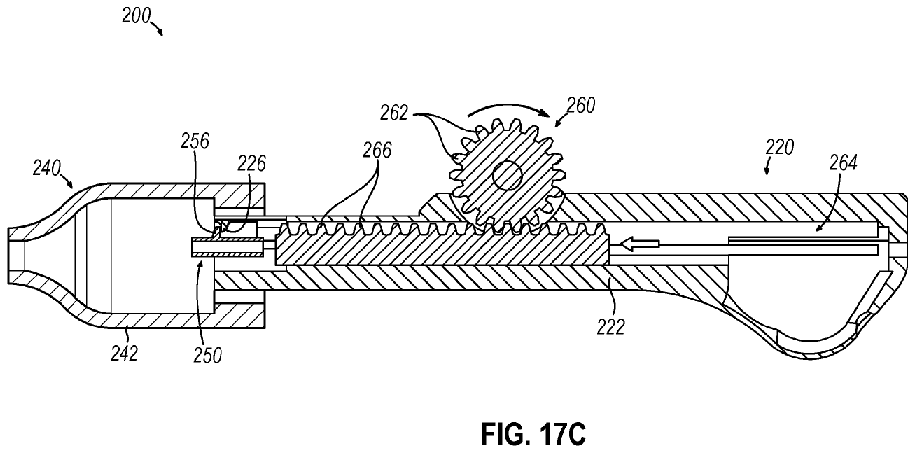

FIG. 16 shows a driver (250), which includes a body (252) with a pair of laterally projecting wings (254) and an upwardly extending latch projection (256). A guide rail (e.g., like guide rails (50, 150)) may be fixedly secured to body (252). Wings (254) are fixedly secured within respective lateral notches (244), such that driver (250) is fixedly secured to guide rail actuator (240). Thus, the guide rail, driver (250), and guide rail actuator (240) may translate unitarily together. Wings (254) are also slidably disposed in lateral slots (224), such that handle (220) supports the combination of driver (250) and guide rail actuator (240) while allowing driver (250) and guide rail actuator (240) to translate relative to handle (220).

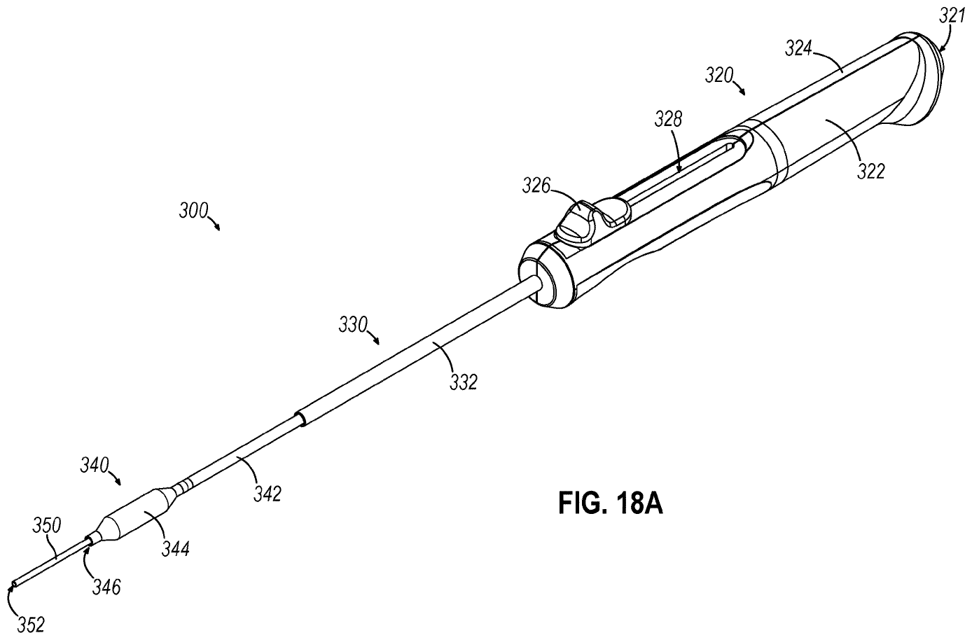

As shown in FIGS. 17A-17C, dilation catheter actuator (260) is in the form of a pinion with teeth (262) that mesh with corresponding teeth (266) of a rack (264). Rack (264) may be coupled with a dilation catheter (e.g., similar to the coupling between rack (164) and dilation catheter (140) described above), such that longitudinal translation of rack (264) provides longitudinal translation of the dilation catheter.

As best seen in FIG. 17A, an operational state of an instrument incorporating body assembly (200) may begin with a dilation catheter and a guide rail both in proximal positions, such that rack (264), driver (250), and guide rail actuator (240) are all in proximal positions. In the present example, driver (250) is positioned to prevent rack (264) from advancing to a fully distal position, such that driver (250) needs to be translated distally to accommodate full distal movement of rack (264). To that end, the operator may urge guide rail actuator (240) distally to the position shown in FIG. 17B. This distal movement of guide rail actuator (240) drives driver (250) and the guide rail distally. The distal positioning of driver (250) provides clearance for rack (264) to advance to a fully distal position.

As driver (250) translates distally from the proximal position shown in FIG. 17A to the distal position shown in FIG. 17B, latch projection (226) interferes with latch projection (256), which is eventually positioned distally relative to latch projection (226). Latch projections (226, 256) thus cooperate to provide a snapping engagement that substantially maintains driver (250) and guide rail actuator (240) in the distal position. In some versions, latch projections (226, 256) are configured to provide a one-way locking engagement such that driver (250) and guide rail actuator (240) may not be retracted back to the proximal position shown in FIG. 17A after reaching the distal position shown in FIG. 17B. In some other versions, latch projections (226, 256) are configured to releasable locking engagement such that driver (250) and guide rail actuator (240) may be retracted back to the proximal position shown in FIG. 17A after reaching the distal position shown in FIG. 17B.

After the guide rail has been advanced distally as shown in the transition from FIG. 17A to FIG. 17B, the operator may advance the dilation catheter distally by rotating dilation catheter actuator (260). This may be accomplished using a finger or thumb of the same hand that grasps handle (220). The rotation of dilation catheter actuator (260) drives rack (264) distally, due to meshing engagement between teeth (262, 266). The distal movement of rack (264) as shown in FIG. 17C drives the dilation catheter distally. While body assembly (200) of the present example includes a rack and pinion actuator arrangement for driving the dilation catheter longitudinally, body assembly (200) may alternatively include a slider actuator or other kind of actuator for driving the dilation catheter longitudinally.

As noted above in the context of instrument (100), there may be scenarios where it is desirable to maintain a guide rail in a proximal position up until an operator is ready to use the instrument. By maintaining the guide rail in the proximal position before use, the positioning of all or nearly all of the distal portion of the guide rail within the dilation catheter may substantially reduce the risk of the distal portion of guide rail getting inadvertently bent, damaged, etc. Such functionality may be achieved through a variation of instrument (100) that includes body assembly (200) just like the functionality being achieved through the version of instrument (100) that includes body assembly (120) as described above.

D. Dilation Instrument with Malleable Guide Rail having Adjustable Longitudinal Position; and Stationary Dilation Catheter

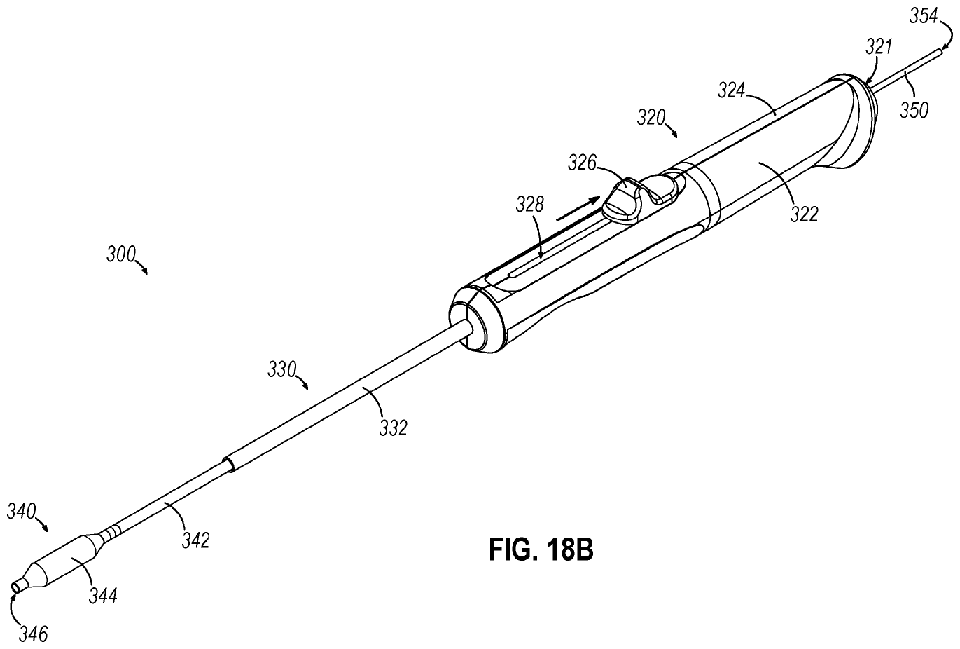

In some scenarios, it may be desirable to provide translation of a guide rail without providing translation of a dilation catheter. This may simplify the construction and operation of the instrument while still achieving the above-noted functionality provided by a guide rail with a distal portion that can be substantially contained within the dilation catheter. To that end, FIGS. 18A-18B show another example of a dilation instrument (300) that may be used to dilate an anatomical passageway in or near an ear, nose, or throat of a patient. Instrument (300) of this example includes a body assembly (320) and a shaft assembly (330). Body assembly (320) includes a first housing (322), a second housing (324), and a dilation catheter actuator (326). Housings (322, 324) cooperate to form a handle. Dilation catheter actuator (326) is slidably disposed in a slot (328) formed in the handle. Dilation catheter actuator (326) is operable to drive longitudinal movement of a guide rail (350) as described in greater detail below.

Shaft assembly (330) includes an outer sheath (332), a guide rail (350), and dilation catheter (340). In the present example, these components are configured to be positioned coaxially with each other, such that outer sheath (332) is positioned externally, dilation catheter (340) is positioned internal to outer sheath (332), and guide rail (350) is positioned internal to dilation catheter (340). Outer sheath (332) of the present example is rigid, though other versions may be malleable or otherwise flexible. In the present example, outer sheath (332) does not enter the head of the patient during operation of instrument (300), though some scenarios may exist where outer sheath (332) enters the patient during operation of instrument (300).

Guide rail (350) of the present example is malleable and has an atraumatic distal tip (352). In some versions, distal tip (352) is dome shaped. In some other versions, distal tip (352) is enlarged (e.g., configured as a ball tip or blueberry tip, etc.). The malleability of guide rail (350) allows guide rail (350) to be bent to a desired bend angle before being inserted into the head of the patient. The malleability of guide rail (350) may allow guide rail (350) to maintain the bend angle of a bend while guide rail (350) is disposed in the head of the patient, including while guide rail (350) is retracted proximally relative to dilation catheter (340). Such operability of guide rail (350) may promote access by dilation catheter (340) to various locations within the head of a patient, such as the maxillary sinus ostium, the frontal recess, the sphenoid sinus ostium, the Eustachian tube, etc., based on the selected bend angle. By way of example only, the bending of guide rail (350) may be performed in accordance with at least some of the teachings of U.S. Pat. No. 11,013,897, entitled “Apparatus for Bending Malleable Guide of Surgical Instrument,” issued May 25, 2021, the disclosure of which is incorporated by reference herein, in its entirety.

Dilation catheter (340) of the present example includes a shaft (342) having an integral balloon (344) and a distal tip (346). Shaft (342) is coaxially positioned with outer sheath (332) and guide rail (350). Shaft (342) defines two inner lumens, including a first lumen in which guide rail (350) is slidably disposed and a second lumen in fluid communication with balloon (344). Balloon (344) may comprise a non-extensible material and may be sized and configured to fit within a targeted anatomical passageway while in the deflated state; then dilate the targeted anatomical passageway while in the inflated state. It should be understood that balloon (344) is shown as being inflated in FIGS. 18A-18B for schematic purposes only; and that balloon (344) may in fact be deflated at such stages of operation. Shaft (342) is fixedly coupled with the handle formed by housings (322, 324), such that dilation catheter (340) does not translate relative to the handle formed by housings (322, 324) in this example.

One or both of distal tips (346, 352) may comprise an indicator element. Each such indicator element may comprise a position sensor and/or an illuminating feature. In versions where an indicator element includes a position sensor, the position sensor may comprise one or more coils that generate signals in response to electromagnetic fields emitted by magnetic field generators of an image guided surgery system that is operable to determine the real-time position of the position sensor in three-dimensional space based on the signals generated by the position sensor. In versions where an indicator element includes an illuminating feature, the illuminating feature may be operable to project light outwardly from indicator element. Such an illuminating feature may provide transillumination through the skin of the patient. In some such versions, the illuminating feature includes an optically transmissive window that is optically coupled with one or more optical fibers, with such one or more optical fibers being optically coupled with a light source. In some other versions, illuminating feature includes one or more LEDs or other local sources of light positioned locally at distal tip (46, 52).

Guide rail actuator (360) is fixedly coupled with guide rail (350) such that guide rail actuator (360) is operable to drive guide rail (350) between the distal position as shown in FIG. 18A and the proximal position shown in FIG. 18B. In operation, the operator may form a bend in the distal portion of guide rail (350) (e.g., the portion of guide rail (350) that is distally exposed relative to dilation catheter (340)) when guide rail is in the distal position. After forming the desired bend, the operator may slide guide rail (350) proximally to the position shown in FIG. 18B. As guide rail (350) translates proximally, the distal portion of dilation catheter (340) may conform to the bend formed in the distal portion of guide rail (350). The malleable properties of the bent distal portion of guide rail (350) may substantially maintain the formed bend angle as the distal portion of dilation catheter (340) traverses the bent distal portion of guide rail (350). By conforming to the bend angle of the bent distal portion of guide rail (350), dilation catheter (340) may be oriented to readily enter the targeted anatomical passageway in the patient.

In the present example, distal tip (352) is distal to distal tip (346) when guide rail (350) is in the distal position; and is proximal to distal tip (346) when guide rail (350) is in the proximal position. In some other versions, distal tip (352) remains distal to distal tip (346) even when guide rail (350) is in the proximal position.

In some scenarios, guide rail (350) is maintained in the proximal position shown in FIG. 18B up until the operator is ready to use instrument (300). By maintaining guide rail (350) in the proximal position before use, the positioning of all or nearly all of the distal portion of guide rail (350) within dilation catheter (340) may substantially reduce the risk of the distal portion of guide rail (350) getting inadvertently bent, damaged, etc. When the operator is ready to use instrument (300), the operator may advance guide rail (350) to the distal position shown in FIG. 18A, then form the desired bend angle in the distal portion of guide rail (350). In some cases, the operator may then insert the distal portion of shaft assembly (330) into the patient (e.g., into the nasal cavity, etc.), then retract guide rail (350) proximally to the position shown in FIG. 18B after the distal portion of shaft assembly (330) is in the patient, then advance the entire instrument (300) such that dilation catheter (340) enters the targeted anatomical passageway. In some other cases, the operator may retract guide rail (350) proximally to the position shown in FIG. 18B before inserting the distal portion of shaft assembly (330) into the patient to thereby position dilation catheter (340) in the targeted anatomical passageway. In either scenario, after dilation catheter (340) is suitably positioned in the targeted anatomical passageway, balloon (344) may be inflated to dilate the targeted anatomical passageway.

### II. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a body assembly; (b) a guide rail extending distally from the body assembly, the guide rail having a malleable distal portion with a distal end; (c) a dilation catheter slidably disposed relative to the guide rail, the dilation catheter including: (i) an expandable element configured to dilate a passageway within a head of a patient, and (ii) a distal end; and (d) a guide rail actuation assembly operable to drive longitudinal movement of the guide rail relative to the body assembly between a distal position and a proximal position, the guide rail actuation assembly being configured to maintain the guide rail at a selected one of the distal position or the proximal position.

### Example 2

The apparatus of Example 1, the body assembly having a proximal end, the guide rail having a proximal end positioned proximally in relation to the proximal end of the body assembly.

### Example 3

The apparatus of any of Examples 1 through 2, at least a portion of the guide rail actuation assembly being positioned proximally relative to the body assembly.

### Example 4

The apparatus of any of Examples 1 through 2, at least a portion of the guide rail actuation assembly being positioned distally relative to the body assembly.

### Example 5

The apparatus of any of Examples 1 through 4, the guide rail actuation assembly being translatable relative to the body assembly between a distal position and a proximal position.

### Example 6

The apparatus of any of Example 5, the guide rail actuation assembly being resiliently based toward the proximal position relative to the body assembly.

### Example 7

The apparatus of any of Examples 5 through 6, further comprising one or more locking features configured to maintain the guide rail actuation assembly in the distal position relative to the body assembly.

### Example 8

The apparatus of Example 7, the one or more locking features being configured to temporarily maintain the guide rail actuation assembly in the distal position relative to the body assembly.

### Example 9

The apparatus of Example 7, the one or more locking features comprising one or more detent features.

### Example 10

The apparatus of Example 7, the one or more locking features comprising one or more latching features.

### Example 11

The apparatus of Example 7 or Example 10, the one or more locking features being configured to permanently maintain the guide rail actuation assembly at the distal position.

### Example 12

The apparatus of any of Examples 1 through 11, the guide rail actuation assembly being further operable to drive rotational movement of the guide rail relative to the body assembly.

### Example 13

The apparatus of Example 12, the guide rail actuation assembly being further operable to selectively lock the angular position of the guide rail relative to the body assembly at one or more selected angular positions.

### Example 14

The apparatus of Example 13, the guide rail actuation assembly comprising one or both of a protrusion or a notch, the body assembly comprising an internal protrusion, the protrusion or notch of guide rail actuation assembly being configured to selectively engage the internal protrusion to thereby selectively lock the angular position of the guide rail relative to the body assembly at a selected angular position.

### Example 15

The apparatus of Example 14, the guide rail actuation assembly being further operable to translate relative to the body assembly between a first longitudinal position and a second longitudinal position.

### Example 16

The apparatus of Example 15, the guide rail actuation assembly being configured to provide engagement of the protrusion or notch of guide rail with the internal protrusion at the first longitudinal position, the guide rail actuation assembly being configured to provide disengagement of the protrusion or notch of guide rail from the internal protrusion at the second longitudinal position.

### Example 17

The apparatus of Example 16, the first longitudinal position being proximal to the second longitudinal position.

### Example 18

The apparatus of any of Examples 1 through 17, the guide rail actuation assembly comprising a knob.

### Example 19

The apparatus of any of Examples 1 through 18, the guide rail actuation assembly comprising a rod.

### Example 20

The apparatus of any of Examples 1 through 19, the guide rail actuation assembly comprising a slider.

### Example 21

The apparatus of any of Examples 1 through 20, the dilation catheter being longitudinally fixed relative to the body assembly.

### Example 22

The apparatus of any of Examples 1 through 20, further comprising a dilation catheter actuation assembly, the dilation catheter actuation assembly being operable to drive longitudinal movement of the dilation catheter along the guide rail.

### Example 23

The apparatus of any of Examples 1 through 22, the guide rail actuation assembly comprising a first translating member, the dilation catheter actuation assembly comprising a second translating member, the first translating member being configured to restrict distal movement of the second translating member when the guide rail is at the proximal position.

### Example 24

The apparatus of Example 23, the second translating member comprising a rack.

### Example 25

The apparatus of Example 24, the dilation catheter actuation assembly further comprising a pinion engaged with the rack, the pinion being rotatable to drive translation of the rack.

### Example 26

A method comprising: (a) forming a bend in a distal portion of a guide rail while the distal portion of the guide rail is exposed relative to a dilation catheter; (b) translating the guide rail longitudinally relative to a body assembly, the guide rail and the dilation catheter extending distally relative to the body assembly; (c) positioning a dilator of the dilation catheter in a targeted anatomical passageway in a patient, at least part of the distal portion of the dilation catheter conforming to the bend in the distal portion of the guide rail during the act of positioning the dilator of the dilation catheter in the targeted anatomical passageway; and (d) expanding a dilator while the dilator is positioned in the targeted anatomical passageway, thereby dilating the targeted anatomical passageway.

### Example 27

The method of Example 26, the act of translating the guide rail longitudinally relative to the body assembly comprising translating the guide rail proximally relative to the body assembly.

### Example 28

The method of any of Examples 26 through 27, the act of translating the guide rail longitudinally relative to the body assembly comprising translating the guide rail distally relative to the body assembly.

### Example 29

The method of any of Examples 26 through 28, targeted anatomical passageway being located in a head of a patient.

### Example 30

The method of Example 29, the targeted anatomical passageway comprising a passageway associated with drainage of a paranasal sinus.

### Example 31

The method of Example 29, the targeted anatomical passageway comprising a Eustachian tube.

### Example 32

The method of any of Examples 26 through 31, further comprising rotating the guide rail relative to the body assembly.

### Example 33

The method of any of Examples 26 through 32, further comprising translating the dilation catheter distally relative to the guide rail, such that a distal portion of the dilation catheter traverses the bend of the guide rail during the act of translating the dilation catheter distally relative to the guide rail.

### Example 34

The method of any of Examples 26 through 33, the act of forming a bend being performed before the act of translating the guide rail longitudinally relative to the body assembly.

### Example 35

The method of any of Examples 26 through 33, the act of forming a bend being performed after the act of translating the guide rail longitudinally relative to the body assembly.

### Example 36

The apparatus of any of Examples 26 through 35, a distal end of the dilation catheter being positioned distally in relation to a distal end of the guide rail during the act of positioning the dilator of the dilation catheter in the targeted anatomical passageway.

### Example 37

An apparatus, comprising: (a) a body assembly; (b) a guide rail, the guide rail having a malleable distal portion with a distal end; (c) a dilation catheter slidably disposed relative to the guide rail, the dilation catheter including: (i) an expandable element configured to dilate a passageway within a head of a patient, and (ii) a distal end; and (d) a guide rail actuation assembly, the guide rail actuation assembly being coupled with the body assembly and movable relative to the body assembly, the guide rail actuation assembly being operable to drive longitudinal movement of the guide rail relative to the body assembly between a distal position and a proximal position, the guide rail actuation assembly being configured to maintain the guide rail at a selected one of the distal position or the proximal position.

### III. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A method for
inducing an immune response in a subject, and/or
reducing one or more clinical signs, viral load and/or viremia caused by a virus infection in a subject,
the method comprising administering orally to the subject an immunogenic composition comprising non-replicating virus antigen.

2. A method for inducing an immune response against PCV2 in a subject, wherein the method comprises the steps of:
adding an immunogenic composition comprising non-replicating PCV2 antigen into the drinking water of the subject or liquid food of the subject, and
allowing the subject to self-administer the immunogenic composition.

3. A method for reducing one or more clinical signs, viral load and/or viremia caused by a virus infection in a subject, wherein the method comprising the steps of:
adding an immunogenic composition comprising non-replicating virus antigen into the drinking water of the subject or the liquid food of the subject, and
allowing the subject to self-administer the immunogenic composition.

4. A method for reducing one or more clinical signs, viral load and/or viremia caused by a PCV2 infection in a subject, wherein the method comprises the steps of:
adding an immunogenic composition comprising non-replicating PCV2 antigen into the drinking water of the subject or the liquid food of the subject, and
allowing the subject to self-administer the immunogenic composition.

5. An immunogenic composition comprising non-replicating virus antigen for use in a method for
inducing an immune response in a subject, and/or
reducing one or more clinical signs, viral load and/or viremia caused by a virus infection in a subject,
wherein the method comprises orally administering said immunogenic composition to said subject.

6. An immunogenic composition comprising non-replicating PCV2 antigen for use in a method for
inducing an immune response against PCV2 in a subject, and/or
reducing one or more clinical signs, viral load and/or viremia caused by a PCV2 infection in a subject,
wherein the method comprises orally administering said immunogenic composition to said subject,

7. The method according to claim 1, or the immunogenic composition for use according to claim 5, wherein said immune response is
an immune response against a virus, wherein said virus is preferably a virus, which is of the species having a genome encoding
the non-replicating virus antigen, or
the amino acid sequence of the antigenic determinant of the non-replicating virus antigen.

8. The method according to any one of claims 1, 3, and 7, or the immunogenic composition for use according to claim 5 or 7, wherein said virus is PCV2; and/or
wherein the immune response is an immune response against PCV2; and/or
wherein said method is a method for reducing one or more clinical signs, viral load and/or viremia caused by a PCV2 infection in a subject.

9. The method according to claim 1 or 3, or the immunogenic composition for use according to claim 5, wherein said virus infection is an infection with a virus, which virus is of the species having a genome encoding
said non-replicating virus antigen, or
the amino acid sequence of the antigenic determinant of said non-replicating virus antigen.

10. The method of any one of claims 7 to 9, or the immunogenic composition for use according to any one of claims 7 to 9, wherein said antigenic determinant is a protein capable of forming a virus-like particle.

11. The method according to any one of claims 1 to 4, and 7 to 10, or the immunogenic composition for use according to any one of claims 5 to 10, wherein the immunogenic composition is free of replicating virus antigen.

12. The method according to any one of claims 1 to 4, and 7 to 11, or the immunogenic composition for use according to any one of claims 5 to 11, wherein the immunogenic composition consists of
said non-replicating virus antigen; and
one or more veterinary-acceptable carriers,
or wherein the immunogenic composition consists of
said non-replicating virus antigen; and
one or more veterinary-acceptable carriers;
and optionally at least one adjuvant and/or optionally at least one immunogenic substance different from said virus antigen.

13. The method according to any one of claims 1 to 4, and 7 to 12, or the immunogenic composition for use according to any one of claims 5 to 12, wherein the immunogenic composition additionally contains a gel composition, in particular a hydrogel composition,
and wherein optionally the gel composition comprises one or more flavoring agents,
and wherein the one or more flavoring agents are preferably capable of attracting pigs,
and/or wherein the one or more flavoring agents are preferably selected from the group consisting of sucrose, glucose, sodium saccharin, sodium cyclamate, xylitol, perillartine, sucralose, D-tryptophan, aspartame, dihydrochalcones, artificial fruit flavoring (e.g., strawberry flavoring), plasma protein (e.g., spray-dried plasma protein), cheese and cheese-like flavorings, dried milk, chocolate and chocolate by-products.

14. The method according to any one of claims 1 to 4, and 7 to 13, or the immunogenic composition for use according to any one of claims 5 to 13, wherein said non-replicating virus antigen comprises or is a recombinant protein,
and wherein the recombinant protein is preferably capable of forming a virus-like particle.

15. The method according to any one of claims 1 to 4, and 7 to 14, or the immunogenic composition for use according to any one of claims 5 to 14 wherein said non-replicating virus antigen is
a virus-like particle, in particular a virus-like particle comprising a recombinant protein.

16. The method according to claim 14 or 15, or the immunogenic composition for use according to claim 14 or 15, wherein the recombinant protein is
a recombinant virus capsid protein, and/or
a recombinant circovirus capsid protein or a recombinant parvovirus capsid protein, and/or
a recombinant porcine circovirus (PCV) capsid protein or a recombinant porcine parvovirus (PPV) capsid protein, and/or
a recombinant porcine circovirus type 2 (PCV2) ORF2 protein,
wherein said PCV2 ORF2 protein comprises or consists of an amino acid sequence having at least 90%, preferably at least 95%, more preferably at least 98%, still more preferably at least 99% or in particular 100% sequence identity with the sequence of SEQ ID NO:1 or SEQ ID NO:2;
and/or wherein said recombinant protein is a recombinant baculovirus expressed protein.

17. The method according to any one of claims 1 to 4, and 7 to 16, or the immunogenic composition for use according to any one of claims 5 to 16, wherein the immunogenic composition comprises
at least 10 μg of the non-replicating virus antigen per dose, or
at least 15 μg of the non-replicating virus antigen per dose, or
at least 30 μg of the non-replicating virus antigen per dose, or
at least 50 μg of the non-replicating virus antigen per dose, or
at least 100 μg of the non-replicating virus antigen per dose, or
at least 150 μg of the non-replicating virus antigen per dose, or
at least 200 μg of the non-replicating virus antigen per dose, or
at least 250 μg of the non-replicating virus antigen per dose.

18. The method according to any one of claims 12 to 17, or the immunogenic composition for use according to any one of claims 12 to 17, wherein said one or more veterinary-acceptable carriers are selected from the group consisting of solvents, dispersion media, coatings, stabilizing agents, diluents, preservatives, antibacterial and antifungal agents, isotonic agents, adsorption delaying agents.

19. The method according to any one of claims 1, 2, and 7 to 18, or the immunogenic composition for use according to aby one of claims 5 to 18, wherein said immune response is a protective immune response.

20. The method according to any one of claims 1, and 7 to 19, or the immunogenic composition for use according to any one of claims 5 to 19, wherein the immunogenic composition is administered by oral drench to the subject.

21. The method according to any one of claims 1 to 4, and 7 to 20, or the immunogenic composition for use according to any one of claims 5 to 20,
wherein the subject is an animal, or
wherein the subject is a pig, or
wherein the subject is a piglet or a sow.

22. The method according to any one of claims 1 to 4, and 7 to 21, or the immunogenic composition for use according to any one of claims 5 to 21, wherein the immunogenic composition comprises 10 to 800 μg of the non-replicating virus antigen per dose.

23. The method according to any one of claims 1 to 4, and 7 to 22, or the immunogenic composition for use according to any one of claims 5 to 22, wherein the immunogenic composition comprises up to 800 μg of the non-replicating virus antigen per dose.

24. A liquid antigen concentrate for dilution in drinking water or in liquid food, wherein 1 mL of said concentrate comprises about 100 to 800 μg of non-replicating virus antigen, preferably about 300 to 800 μg of non-replicating virus antigen.

25. A method for enabling the self-immunization against a virus by a subject, comprising the step of diluting a liquid antigen concentrate comprising non-replicating virus antigen in the drinking water of the subject.

26. Container containing drinking water to which a liquid antigen concentrate comprising non-replicating virus antigen has been added.

27. The container according to claim 26, wherein said container is selected from the group consisting of
a bowl containing drinking water to which a liquid antigen concentrate comprising non-replicating virus antigen has been added,
a vessel containing drinking water to which a liquid antigen concentrate comprising non-replicating virus antigen has been added, and
a barrel containing drinking water to which a liquid antigen concentrate comprising non-replicating virus antigen has been added;
and/or wherein said container is located in the housing environment of a subject to be immunized.

28. Kit for immunization of a subject, comprising
a liquid antigen concentrate comprising non-replicating virus antigen; and
instructions to dilute said liquid antigen concentrate in drinking water or liquid food to be consumed by the subject.

29. The liquid antigen concentrate for dilution in drinking water of claim 24, the method according to claim 25, the container according to claim 26 or 27, or the kit according to claim 28, wherein said non-replicating virus antigen is a non-replicating virus antigen as specified in claim 14 or 15, and/or wherein said non-replicating virus antigen comprises a recombinant protein as specified in claim 16.

30. The method according to claim 25 or 29, the container according to any one of claims 26, 27, and 29, or the kit according to claim 28 or 29, wherein said liquid antigen concentrate comprising non-replicating virus antigen is the liquid antigen concentrate of claim 24 or 29.

31. The method according to any one of claims 25, 29, and 30, the container according to any one of claims 27, 29, and 30, or the kit according to any one of claims 28 to 30,
wherein the subject is an animal, or
wherein the subject is a pig, or
wherein the subject is a piglet or a sow.
